(19) United States
(12) Patent Application Publication
(10) Pub. No.: US 2012/0226185 A1
(43) Pub. Date: Sep. 6, 2012

(54) READINESS POTENTIAL-BASED BRAIN-COMPUTER INTERFACE DEVICE AND METHOD

(75) Inventors: Chun Kee CHUNG, Seoul (KR); June Sic KIM, Seoul (KR); Kyung In CHOI, Seoul (KR); Hong Gi YEOM, Seoul (KR)

(73) Assignee: SEOUL NATIONAL UNIVERSITY R&DB FOUNDATION, Seoul (KR)

(21) Appl. No.: 13/274,342

(22) Filed: Oct. 16, 2011

(30) Foreign Application Priority Data
Mar. 3, 2011 (KR) 10-2011-0019130

(51) Int. Cl. A61B 5/0476

(52) U.S. Cl. 600/544

(57) ABSTRACT

The present invention provides a brain-computer interface device. The brain-computer interface device may include: a preprocessor for preprocessing a readiness potential signal measured by a brain wave detection device; a noise eliminator for eliminating noise from the preprocessed readiness potential signal; a signal processor for extracting features related to a user's intention by calculating at least one of the intensity of the readiness potential signal from which noise is eliminated, the phase of the readiness potential signal, the place where the readiness potential signal is generated, and the time when the readiness potential signal is generated; and a data classifier for classifying the extracted features to determine the user's intention.

### CROSS-REFERENCE TO RELATED PATENT APPLICATION

This application claims the benefit of Korean Patent Application No. 10-2011-0019130, filed on Mar. 3, 2011, in the Korean Intellectual Property Office, the disclosure of which is incorporated herein in its entirety by reference.

### BACKGROUND OF THE INVENTION

1. Field of the Invention

The present invention relates to a brain-computer interface and, more particularly, to a readiness potential-based brain-computer interface device and method.

2. Description of the Related Art

A brain-computer interface (BCI), which allows a direct connection between brain and computer, is one of the new human computer interfaces that convert a person's will or thought, formed by a group of neurons that constitute the brain, into a digital signal recognizable by a computer. While the communication with the digital world that takes place on a network becomes as important as the communication with the physical world that takes place through the human body, the desire of users to use the computers more equally, conveniently and freely becomes stronger.

The BCI technology is a technology that moves a mouse cursor or controls a robot only by thought and can be conveniently used by a paralyzed patient who cannot move, and thus the BCI technology is very useful and can be used anywhere.

Various technologies for measuring brain activity have been developed based on the fact that neuronal signaling pathways have electrical and chemical properties. The technologies for measuring brain activity include electroencephalography (EEG), magnetoencephalography (MEG) which detects magnetic fields from the brain, magnetic resonance imaging (MRI) which measures the density of hydrogen atoms using the magnetic fields from the brain, positron emission tomography (PET) which examines functional aspects of the brain by injecting a radioactive chemical into blood vessels, functional magnetic resonance imaging (fMRI) which analyzes the functional activity of the brain by measuring changes in blood flow occurring during brain activity, etc. (KIM Dae-sik, CHOI Jang-wook, 2001; LEE Jung-mo, etc. 2003; Stafford, Webb, 2004).

According to KIM Dae-sik, in the case of MRI or PET, it is possible to measure the brain activity spatially, but the temporal resolution is lower than that of MRI and PET. In the case of EEG, it is cheaper than MEG and can identify changes in brain activity both temporally and spatially, and there is no significant difference in analysis results.

Therefore, extensive research on the brain-computer interface which controls a device based on EEG analysis has continued to progress. Prior research on the brain-computer interface using EEG will be described below. The possibility of controlling a control using EEG has been confirmed by research on the “Mind Switch” carried at the University of Technology in Sydney, which turns a switch on and off based on a reaction in which the alpha waves increase in a relaxed state with closed eyes and are reduced with open eyes and by research on the cursor control and character/word selection for disabled people carried at the Technische Universitat Graz in Austria (EUM Taekwan, KIM Eung-su, recited in 2004).

When the EEG is used, people with speech disorders and patients or disabled people with paralysis can easily control devices such as computers only by their thoughts with their own intentions (Wolpaw, Birbaumer, McFarland, Pfurtscheller, & Vaughan, 2002). Further, extensive research aimed at utilizing EEG in various entertainment environments has continued to progress, and research aimed at playing 3D games using EEG has been carried out at the California State University (Pineda, Silverman, Vankov, & Hestenes, 2003).

Meanwhile, the brain-computer interface technologies for the use of EEG can be generally classified into two categories such as “invasive methods” and “non-invasive methods”.

The invasive method measures signals directly from the brain in the skull through surgery, for example, and the non-invasive method obtains signals from the surface of the scalp.

The invasive method has the advantages that the noise is small and an accurate signal can be obtained from a narrower area but has the disadvantage that the surgery is required. On the contrary, the non-invasive method can be applied to ordinary people without the need of surgery but has the disadvantage that the signal distortion increases.

At present, extensive research aimed at providing a faster and more accurate brain-computer interface by the non-invasive method has continued to progress such that many people can conveniently use the brain-computer interface.

However, since the brain does different things at the same time, it is important to extract features that better reflects the user's intention. In the case of the non-invasive method, since the signal distortion is large, it is important to extract a related signal from the brain by minimizing the signal distortion and removing the noise.

This technique is called feature extraction, which extracts only important and necessary information from a large amount of EEG signal data measured from the brain, and can be considered as the core of the brain-computer interface technology.

Research on the existing brain-computer interfaces can be generally classified into four categories based on the types of EEG signals such as slow cortical potential (SCP), sensorimotor rhythm (SMR), P300, and steady-state visually evoked potential (SSVEP). The slow cortical potential is a signal which varies depending on the synchronicity and intensity of the afferent input to cortical layers I and II and thus the response is very slow. The sensorimotor rhythm is related to the increase and decrease in mu waves or beta waves over the sensorimotor cortex, which also uses the signal after movement and thus the response of the interface is slow. Moreover, the P300 and steady-state visually evoked potential are also related to the interface technology, which uses a signal elicited by a given stimulus, and thus are inconvenient to use due to temporal delay.

### SUMMARY OF THE INVENTION

The present invention has been made in an effort to solve the above-described problems associated with prior art, i.e., the problem that the response of an existing brain-computer interface is slow. In detail, an abject of the present invention is to solve the above-described problem based on the fact that during voluntary movement, a readiness potential is generated before the movement, while it varies from person to person. In more detail, the present invention uses the fact that during voluntary movement, a fast readiness potential is generated at −2,000 ms to −1,500 ms before the movement and a slow readiness potential is generated at −500 ms to 0 ms.

In order to achieve the above-described objects of the present invention, the present invention provides a brain-computer interface technology which recognizes a user's intention before movement using a readiness potential. In detail, the present invention provides a technology for analyzing a user's intention before movement using a readiness potential and providing a service such that the user can control a computer or machine in real time without feeling any inconvenience.

To achieve the above object of the present invention, the present invention may provide a brain-computer interface device. The brain-computer interface device may include: a preprocessor for preprocessing a readiness potential signal measured by a brain wave detection device; a noise eliminator for eliminating noise from the preprocessed readiness potential signal; a signal processor for extracting features related to a user's intention by calculating at least one of the intensity of the readiness potential signal from which noise is eliminated, the phase of the readiness potential signal, the place where the readiness potential signal is generated, and the time when the readiness potential signal is generated; and a data classifier for classifying the extracted features to determine the user's intention.

The preprocessor may include at least one selected from the group consisting of a low-pass filter, a high-pass filter, and a band-pass filter.

The noise eliminator may perform at least one of independent component analysis (ICA) to remove noise mixed with the readiness potential signal and principal component analysis (PCA) to remove noise mixed with the readiness potential signal and to extract only the readiness potential signal.

The signal processor may extract features related to a user's intention by calculating at least one of the intensity of the readiness potential signal from which noise is eliminated, the phase of the readiness potential signal, the place where the readiness potential signal is generated, and the time when the readiness potential signal is generated.

The data classifier may perform a classification algorithm such as neural networks, support vector machine (SVM), bayesian networks, linear discriminant analysis (LDA), etc.

The brain-computer interface device may receive the classified information and determine the user's intended operation based on the classified information.

To achieve the above object of the present invention, the present invention may provide a brain-computer interface method. The brain-computer interface method may include preprocessing a readiness potential signal measured by a brain wave detection device; eliminating noise from the preprocessed readiness potential signal; extracting features related to a user's intention by calculating at least one of the intensity of the readiness potential signal from which noise is eliminated, the phase of the readiness potential signal, the place where the readiness potential signal is generated, and the time when the readiness potential signal is generated; classifying the extracted features to determine the user's intention; and controlling the operation of a computer by determining the user's intended operation based on the classified information.

In the eliminating the noise, independent component analysis (ICA) may be performed to remove noise mixed with the readiness potential signal.

In the eliminating the noise, principal component analysis (ICA) may be performed to remove noise mixed with the readiness potential signal and to extract only the readiness potential signal.

### BRIEF DESCRIPTION OF THE DRAWINGS

The above and other features and advantages of the present invention will become more apparent by describing in detail exemplary embodiments thereof with reference to the attached drawings in which:

FIG. 1 shows the structure of neurons;

FIG. 2 is a diagram showing the types of brain waves according to frequency bands;

FIG. 3 is a diagram showing the structure and function of the brain;

FIG. 4 is a diagram showing the arrangement of electrodes on the head for measurement of brain waves;

FIG. 5 is a diagram showing a difference in readiness potential according to a user's intention;

FIG. 6 is a diagram showing the configuration of a system according to the present invention; and

FIG. 7 is a block diagram showing the configuration of an interface device according to the present invention.

### DETAILED DESCRIPTION OF THE INVENTION

Hereinafter, embodiments of the present invention will be described in detail with reference to the accompanying drawings. Like reference numerals in the drawings denote like elements, and thus repeated descriptions will be omitted. While the accompanying drawings are provided to more clearly describe the features of the present invention, it will be understood by those skill in the art that the scope of the present invention should not be construed as limited to those of the accompanying drawings.

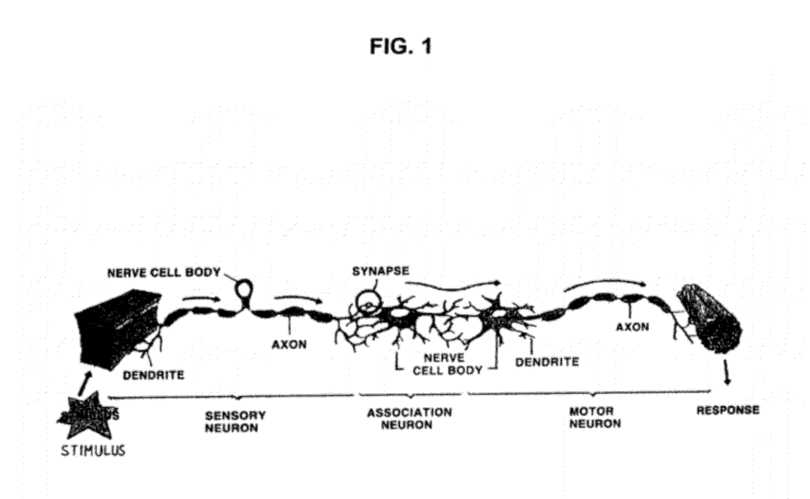

FIG. 1 shows the structure of neurons.

A nervous system that facilitates both physical and mental activities consists of nerve cells, and the basic unit of the nervous system is a neuron. As shown in FIG. 1, the neuron, the smallest nerve cell, consists of a cell body, a dendrite, and an axon and functions to transmit information from and to other cells. The neuron transmits signals between nerve cells by electrical signals transmitted by changes in osmotic pressure and electrical potential across the cell membrane.

The types of neurons include a sensory neuron, an association neuron, and a motor neuron. The sensory neuron functions to transmit a stimulus received by a sensory organ, and the motor neuron functions to transmit a decision or command of the central nervous system to a muscle or effector. The association neuron functions to connect the motor neuron and the sensory neuron. The human brain consists of about 100 billion neurons, and brain waves are produced due to differences in electrical potential when synapses, which are connections between neurons, transmit information.

Hans Berger who first measured and recorded brain waves developed the electroencephalogram (EEG). Among various methods for analyzing the brain waves measured by EEG, a classification system for frequency bands, which was first used and proposed by Berger, has been widely used.

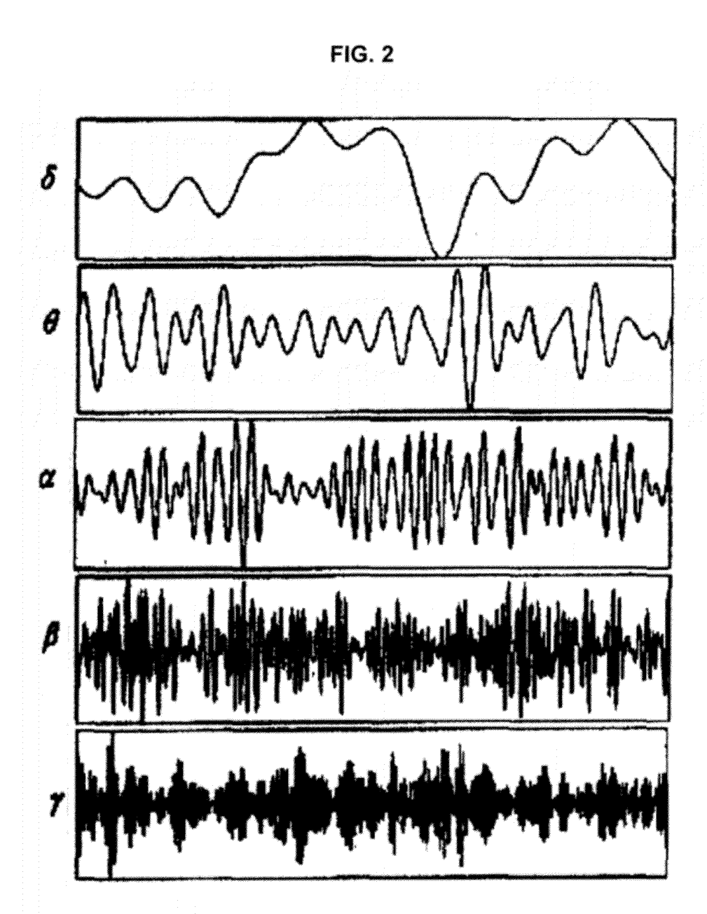

FIG. 2 is a diagram showing the types of brain waves according to frequency bands.

The brain waves are represented by the period, frequency, and amplitude. Typically, the brain waves have a frequency of 1 to 60 Hz and an amplitude of about 5 to 300 μV. The frequency is widely used in brain-wave reading, instead of the period. The brain waves exhibits different characteristics according to frequency bands and can be classified as gamma (γ) waves, beta (β) waves, alpha (α) waves, theta (θ) waves, and delta (δ) waves as shown in the following table 1:

TABLE 1

| Type | Frequency (Hz) | Characteristics |
|---|---|---|
| Gamma (γ) waves | 30 or higher | Produced during extreme vigilance |
| | | or excitement |
| | | Produced most frequently in the |
| | | frontal lobe and parietal lobe |
| Beta (β) waves | 13 to 30 | Fast waves produced in normal adults |
| | | during excitement or tension |
| | | Produced when attention is needed |
| | | and during intense mental activity |
| Alpha (α) waves | 8 to 13 | Stable waves produced in normal |
| | | adjusts during relaxation |
| | | Inversely related to the mental |
| | | activity (reduced when attention |
| | | is needed) |
| Theta (θ) wave | 4 to 8 | Produced most frequently in a sleep |
| | | or meditative state |
| | | Related to body or emotion |
| | | associated with a deeply internalized |
| | | state and with a quieting of the body |
| Delta (δ) waves | 2 to 4 | Prevailing in a sleep state where the |
| | | brain function is completely lost |
| | | Produced in patients with brain |
| | | tumor, encephalitis, mental disease, |
| | | etc. |

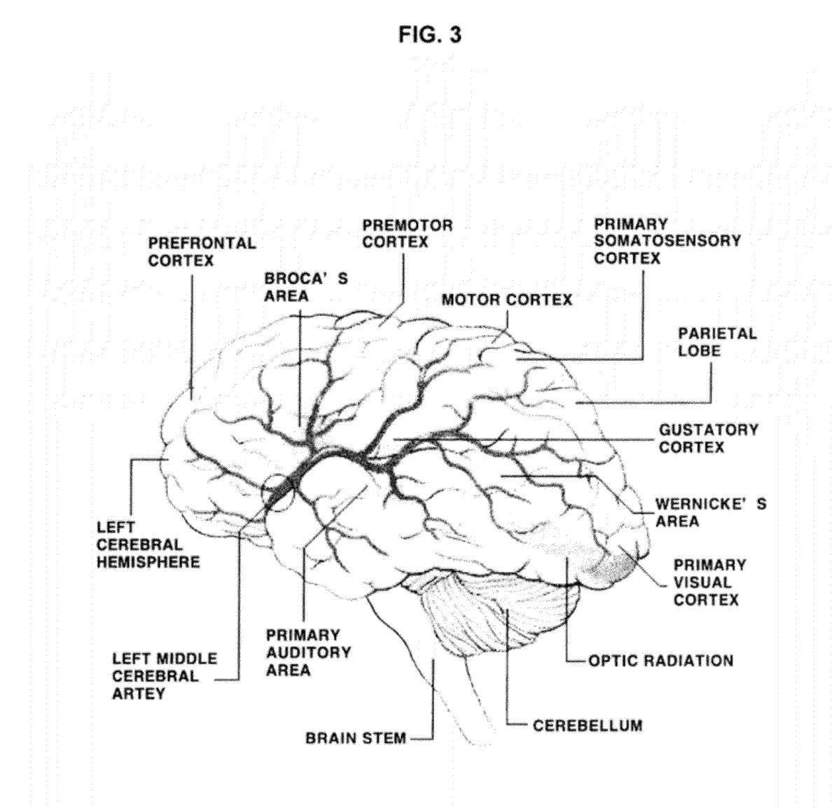

FIG. 3 is a diagram showing the structure and function of the brain.

The human brain consists of the cerebrum, the cerebellum, and the brain stem. To measure brain waves by non-invasive EEG, electrodes are located on the scalp. The brain waves are much affected by the cerebral cortex closest to the scalp.

The cerebral cortex occupies a large portion of the brain and is the area of the brain that is most developed in human beings. The cerebral cortex is responsible for motor, sensory, and association functions. The motor function of the cerebral cortex involves all muscle movements, and the sensory function of the cerebral cortex involves all human senses such as sight, hearing, smell, taste, tough, etc. The association function of the cerebral cortex involves the human's higher mental functions such as rational thought, language, higher order thinking, etc.

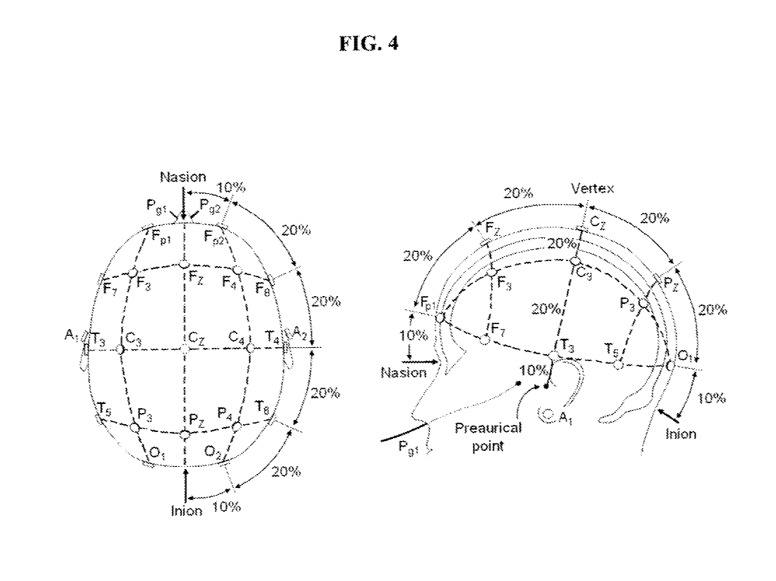

FIG. 4 is a diagram showing the arrangement of electrodes on the head for measurement of brain waves.

The brain waves are generally referred to as scalp EEG captured from scalp electrodes. However, in addition to the brain waves, there are several kinds of EEG recording methods such as electrocorticography (ECoG), sphenoidal electrode EEG, foramen ovale electrode EEG, depth electrode EEG, etc. according to the type of electrodes used and the installation method. Of course, according to the type of electrodes used, the area to be examined by EEG and the purpose of EEG recording are diversified. Obviously, it is necessary to select and use appropriate electrodes depending on the purpose of medical treatment and to select the area on which the electrodes are to be located and the type of electrodes used depending on the purpose of basic medical research during EEG recording. Typically, the location of scalp electrodes is based on the international 10-20 system shown in FIG. 4.

The international 10-20 system is the most widely used method to describe the location of scalp electrodes, and the location of scalp electrodes is shown in FIG. 4. In FIG. 4, the alphabetic letters represent the frontal, central, parietal, temporal, and occipital, respectively, and Fp represents the frontopolar. FIG. 4 is an image taken from the top of the head, in which the electrodes are placed in a ratio of 20, 20, and 10, respectively, when the ratio between the electrodes from the calvaria to the nasion, from the calvaria to the inion, and from the calvaria to the top of the pinna is 50, respectively. According to this description, the image viewed from the left side is symmetrical to that viewed from the right side. The international 10-20 system for EEG electrode placement has been widely used for a long time.

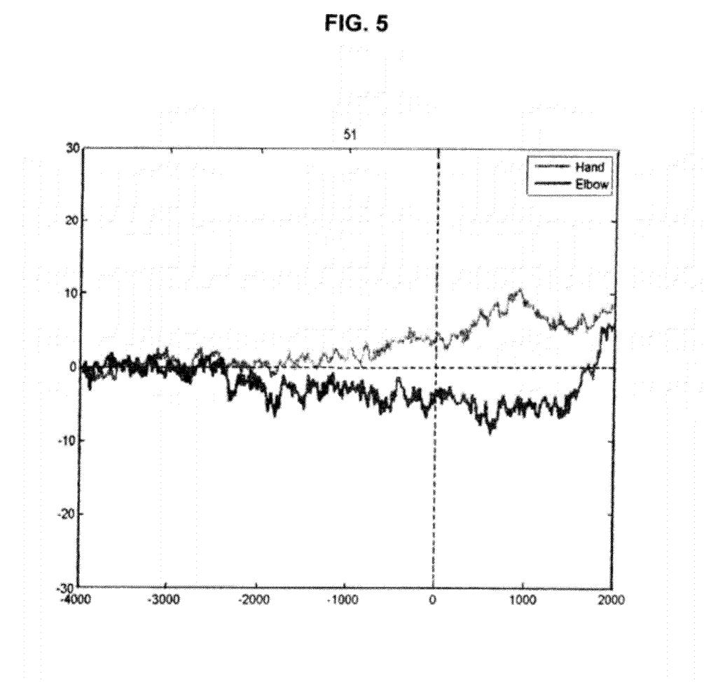

FIG. 5 shows the recording of readiness potential.

As shown in FIG. 5, it can be seen that during voluntary movement, a fast readiness potential is generated at −2,000 ms to −1,500 ms before the movement and a slow readiness potential is generated at −500 ms to 0 ms, while the readiness potential varies from person to person.

The red line shown in FIG. 5 represents the readiness potential generated when the hand is to be moved and the blue line represents the readiness potential generated when the elbow is to be moved.

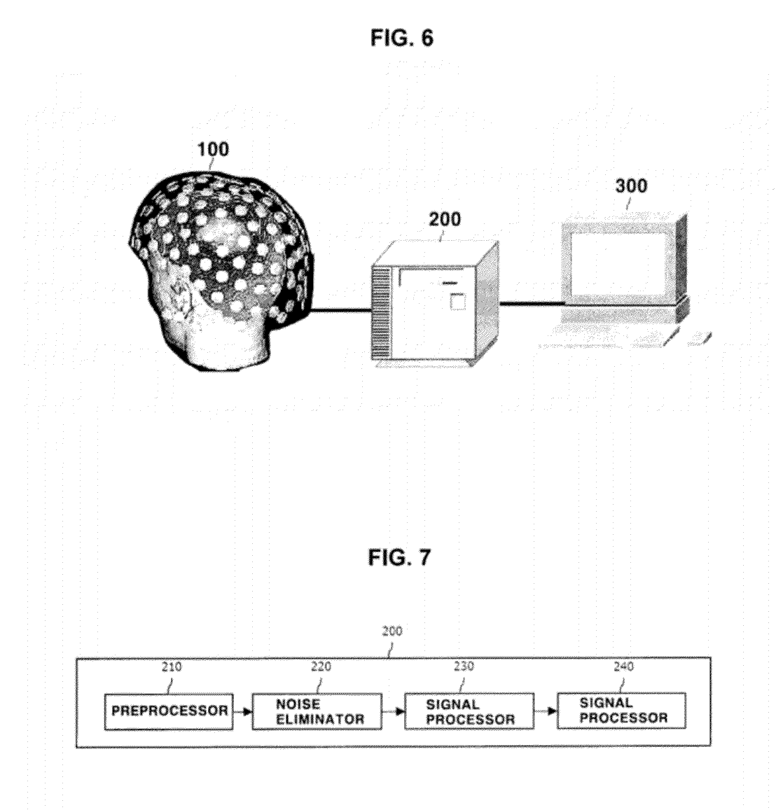

FIG. 6 is a diagram showing the configuration of a system according to the present invention, and FIG. 7 is a block diagram showing the configuration of an interface device according to the present invention.

As can be seen with reference to FIG. 6, the system according to the present invention includes a brain wave detection device 100, an interface device 200, and a computer 300.

The brain wave detection device 100 may detect a readiness potential by any one of non-invasive methods such as electroencephalography (EEG), magnetoencephalography (MEG), near-infrared spectroscopy (NIRS), etc. and invasive methods such as micro electrode, electrocorticography (ECoG), etc.

The interface device 200 interfaces with a head of an animal, including a human, and the computer 300 through a readiness potential measured by the brain wave detection device 100 placed on the head of the animal.

The configuration of the above-described interface device 200 will now be described in more detail with reference to FIG. 7.

The interface device 200 may include a preprocessor 210 for preprocessing the readiness potential signal measured by the brain wave detection device 100, a noise eliminator 220 for eliminating noise, a signal processor 230, and a data classifier 240.

The preprocessor 210 performs a preprocessing process for feature extraction and noise elimination, because the frequency characteristics are difference between individuals. The preprocessor 210 may include at least one of a low-pass filter, a high-pass filter, and a band-pass filter, each having a different band for each user.

Moreover, the preprocessor 210 may include a notch filter for reducing noise due to a power line, a reference voltage changing unit (or referencing unit), a normalization unit, and a base-line correction unit to minimize the difference between users and the difference in a user.

Meanwhile, the noise eliminator 220 may perform independent component analysis (ICA), principal component analysis (PCA), etc. Noise such as electromyogram (EMG), electrooculogram (EOG), etc. can be eliminated by the noise eliminator 220.

The independent component analysis (ICA) is to remove noise mixed with brain waves, and the noise may be generated by movement of the neck, face, and eyes. Accordingly, a subject's attention is needed during the measurement of brain waves. Despite the subject's attention, noise in brain areas adjacent to the subject area, which is functionally separated from the adjacent brain areas, may be mixed with the brain waves to be measured. Thus, the unnecessary noise can be separated from the mixed signal by the ICA.

In detail, the ICA is to extract original signals from the resulting signals in which several signals are mixed together. The ICA is one of blind source separation (BSS) methods to extract source signals by analyzing the results obtained only by measurement, even without the information on the source location or route of linear signals. For example, in the case of data recorded when two people talk at the same time, the two people's voices can be separated from the recorded data. The ICA can analyze stochastically independent signals by minimizing the correlation and dependency between several signals and maximizing the entropy.

Since the brain waves are a combination of linear signals measured by multiple electrodes, the source of neural activity cannot be accurately identified. Thus, it is possible to extract the signal closest to the original signal using the ICA. In brain wave research, the ICA is used to separate several independent signals from the brain waves measured from multiple electrodes.

Meanwhile, the signal processor 230 extracts features related to a user's intention by calculating the intensity of readiness potential, the phase of the readiness potential signal, the place where the readiness potential signal is generated, the time when the readiness potential signal is generated, etc. The signal processor 230 may perform Fourier transform for detecting frequency components or brain signal source localization for detecting the place where the readiness potential is generated and may calculate the intensity of readiness potential, the change in signal intensity, the power of signal, etc.

The data with the extracted features is input to the data classifier 240. The data classifier 240 may perform a classification algorithm such as neural networks, support vector machine (SVM), bayesian networks, linear discriminant analysis (LDA), etc.

As such, if the extracted features are classified to determine the user's intention by the data classifier 240, the classified information is input to the computer 300. Then, the computer 300 performs the user's intended operation. For example, if the classified information is the movement of the user's index finger, the computer 300 can move a mouse pointer to the left. Otherwise, if the classified information is the movement of the user's middle finger, the computer 300 can move the mouse pointer to the right.

As described above, the present invention can solve the above-described problem that the response of the existing brain-computer interface is slow. Moreover, the present invention analyzes a user's intention before movement using a readiness potential and provides a service such that the user can control a computer or machine in real time without feeling any inconvenience.

While the invention has been particularly shown and described with reference to exemplary embodiments thereof, it will be understood by those of ordinary skill in the art that various changes in form and details may be made therein without departing from the spirit and scope of the invention as defined by the following claims.

What is claimed is:

1. A brain-computer interface device comprising:
a preprocessor for preprocessing a readiness potential signal measured by a brain wave detection device;
a noise eliminator for eliminating noise from the preprocessed readiness potential signal;
a signal processor for extracting features related to a user's intention by calculating at least one of the intensity of the readiness potential signal from which noise is eliminated, the phase of the readiness potential signal, the place where the readiness potential signal is generated, and the time when the readiness potential signal is generated; and
a data classifier for classifying the extracted features to determine the user's intention.

2. The brain-computer interface device of claim 1, wherein the preprocessor comprises at least one selected from the group consisting of a low-pass filter, a high-pass filter, and a band-pass filter.

3. The brain-computer interface device of claim 1, wherein the noise eliminator performs independent component analysis (ICA) to remove noise mixed with the readiness potential signal.

4. The brain-computer interface device of claim 1, wherein the noise eliminator performs principal component analysis (PCA) to remove noise mixed with the readiness potential signal and to extract only the readiness potential signal.

5. The brain-computer interface device of claim 1, further comprising a computer device for receiving the classified information and determining the user's intended operation based on the classified information.

6. A brain-computer interface method comprising:
preprocessing a readiness potential signal measured by a brain wave detection device;
eliminating noise from the preprocessed readiness potential signal;
extracting features related to a user's intention by calculating at least one of the intensity of the readiness potential signal from which noise is eliminated, the phase of the readiness potential signal, the place where the readiness potential signal is generated, and the time when the readiness potential signal is generated;
classifying the extracted features to determine the user's intention; and
controlling the operation of a computer by determining the user's intended operation based on the classified information.

7. The brain-computer interface method of claim 6, wherein in the eliminating the noise, independent component analysis (ICA) is performed to remove noise mixed with the readiness potential signal.

8. The brain-computer interface method of claim 6, in the eliminating the noise, principal component analysis (PCA) is performed to remove noise mixed with the readiness potential signal and to extract only the readiness potential signal.
